(19) United States
(12) Patent Application Publication
(10) Pub. No.: US 2026/0237473 A1
(43) Pub. Date: Aug. 13, 2026

(54) Audio Data Signal Processing for Automated Speech Recognition Pipelines

(71) Applicant: Oracle International Corporation, Redwood Shores, CA (US)

(72) Inventors: Meizhu Liu, Livingston, NJ (US); Daniel Bruce Carter, Redmond, WA (US)

(73) Assignee: Oracle International Corporation, Redwood Shores, CA (US)

(21) Appl. No.: 19/235,980

(22) Filed: Jun. 12, 2025

Related U.S. Application Data
(60) Provisional application No. 63/757,217, filed on Feb. 11, 2025.

(51) Int. Cl. G16H 10/60; G10L 15/06; G10L 15/28

(52) U.S. Cl. CPC G16H 10/60; G10L 15/063; G10L 15/28

(57) ABSTRACT

A machine learning (“ML”) pipeline for automated speech recognition (“ASR”) is disclosed. The pipeline accesses source audio data including digital recordings of one or more voices. The pipeline performs offline digital signal processing of the source audio data, at least by enriching the source audio data with an added audio element, to obtain enriched audio data. The pipeline trains an ML model using the enriched audio data as training data. The pipeline applies the ML model to unlabeled audio data, to obtain a transcription of the unlabeled audio data.

### INCORPORATION BY REFERENCE; DISCLAIMER

Each of the following applications are hereby incorporated by reference: Application No. 63/757,217 filed on Feb. 11, 2025. The applicant hereby rescinds any disclaimer of claims scope in the parent application(s) or the prosecution history thereof and advises the USPTO that the claims in the application may be broader than any claim in the parent application(s).

### TECHNICAL FIELD

The present disclosure relates to automated speech recognition (“ASR”). In particular, the present disclosure relates to improving the performance of ASR systems.

### BACKGROUND

Machine learning (“ML”) models learn patterns from data and make predictions or decisions based on the learned patterns. ASR is a specific application of ML that transforms spoken language into text using language models trained on speech and language data. ASR receives a live audio stream or voice recording and generates a written transcript of spoken language detected in the audio data. ASR may be used in transcription applications, voice assistants, chatbots, etc.

ASR faces various technical challenges when dealing with recordings that have unfamiliar words, voices with different accents, background noise, and/or other complicating factors. These technical challenges include, but are not limited to, the following:
1. Some environments deal with private and/or legally protected data that cannot ethically and/or legally be used to train ASR models. For example, in the United States, the Health Insurance Portability and Accountability Act (“HIPAA”) would protect recordings of medical appointments. Even if a provider wanted to make patient data available to train ASR models, they would be legally barred from doing so without obtaining a release from the patient. These hurdles make it prohibitively challenging to amass a sufficiently large corpus of audio recordings from such environments to use as training data. In these situations, ASR model training relies on smaller and/or less relevant training data (e.g., audio data from other environments). ASR generally does not perform as well when highly relevant training data is sparse or lacking.
2. In some contexts, vocabulary changes and/or expands rapidly. For example, technical vocabulary is continually expanding with new terms. Examples of technical vocabulary include species names, new drug names, medical conditions, scientific terms, etc. ASR generally does not perform as well at transcribing unfamiliar words.
3. Voices may present with unfamiliar accents and/or speech patterns. For example, in a medical setting, patients may have accents and/or speech patterns affected by health conditions. ASR generally does not perform as well in environments with unfamiliar accents and/or speech patterns.
4. Some environments introduce significant amounts of background noise. For example, in a medical setting, background noise may include sounds produced by medical equipment, background voices (e.g., in a busy hospital), sirens, etc. In a street setting, background noise may include sounds produced by cars, construction equipment, etc. ASR generally does not perform as well when background noise obscures the voice(s) being transcribed.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates an ASR system in accordance with one or more embodiments;

FIG. 2 illustrates an example set of operations for ASR in accordance with one or more embodiments;

FIGS. 3A-E illustrate an example of ASR in accordance with one or more embodiments;

FIG. 4 illustrates a machine learning engine in accordance with one or more embodiments;

FIG. 5 illustrates the operation of a machine learning engine in one or more embodiments; and

FIG. 6 shows a block diagram that illustrates a computer system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.
1. GENERAL OVERVIEW
2. PRACTICAL APPLICATIONS, ADVANTAGES, AND IMPROVEMENTS
3. EXAMPLE ASR PIPELINE
4. EXAMPLE OPERATIONS FOR ASR
5. EXAMPLES OF ASR
   5.1. SOURCE AUDIO DATA TRANSCRIPTION
   5.2. HIGH CONFIDENCE AUDIO DATA ENRICHMENT
   5.3. EXTREME CONFIDENCE AUDIO DATA ENRICHMENT
   5.4. MACHINE LEARNING USING ENRICHED HIGH CONFIDENCE AUDIO DATA
   5.5. MACHINE LEARNING USING ENRICHED EXTREME CONFIDENCE AUDIO DATA
   5.6. ELASTIC WEIGHT CONSOLIDATION-BASED LOSS FUNCTION
6. MACHINE LEARNING ARCHITECTURE
7. MACHINE LEARNING OPERATIONS
8. GENERATIVE ARTIFICIAL INTELLIGENCE MODELS
9. COMPUTER NETWORKS AND CLOUD NETWORKS
10. MICROSERVICE APPLICATIONS
11. HARDWARE OVERVIEW
12. MISCELLANEOUS; EXTENSIONS

### 1. General Overview

One or more embodiments execute an ML training pipeline that filters and enriches training data to improve model robustness. Specifically, one or more embodiments execute an ML training pipeline that improves speech recognition and system performance using one or more of the following:

Enriching training data. One or more embodiments enrich training data to enhance diversity of the training data and improve model robustness. Specifically, one or more embodiments enrich training data using techniques such as noise injection, voice cloning (replacing voices in labeled data with different voices), introducing sound effects (e.g., medical background sounds, traffic sounds, stadium sounds, auditorium sounds), changing audio speed, etc.

Generating weak supervision signals. One or more embodiments transcribe unlabeled audio data and estimate, for resulting tokens, a probability that the transcription is accurate. The overall confidence score for a transcription is the average of probabilities across tokens.

Data filtering. One or more embodiments use high-confidence transcription pairs (audio and transcriptions) as training data.

Loss functions to preserve knowledge. To prevent losing knowledge from one iteration of the ML model to the next, one or more embodiments incorporate one or more loss functions.

Adaptive training. One or more embodiments use a multi-layer ML model in which earlier layers capture a broad understanding of the audio and later layers focus on task-specific adaptations. One or more embodiments fine-tune the entire model using very high-confidence audio-transcription pairs. Additionally, or alternatively, one or more embodiments selectively fine-tune later layers using additional high-confidence audio-transcription pairs.

One or more embodiments described in this Specification and/or recited in the claims may not be included in this General Overview section.

### 2. Practical Applications, Advantages, and Improvements

One or more embodiments provide technical solutions to one or more of the technical problems described herein. Specifically, embodiments provide one or more of the following technical improvements:
1. One or more embodiments use training data enrichment to train an ASR model that is more robust to unfamiliar words, voices with different accents, background noise, and/or other complicating factors. Training data enrichment (e.g., to introduce noise, modify speaker voices, introduce relevant sound effects, change audio speed, etc.) allows the model to be trained using audio data that more closely approximates the target environment, even when a sufficiently large corpus of audio recordings from the target environment is not available to use as training data.
2. One or more embodiments use weak signal generation to obtain labeled training data even when manual ground-truth transcriptions are not available. Obtaining manual ground-truth transcriptions is often prohibitively time-consuming and/or expensive, especially in environments where people frequently use specialized and/or technical terminology (e.g., medical environments, science laboratory environments, etc.) that requires transcribers to have domain-specific knowledge. In many cases, obtaining manual ground-truth transcriptions cannot realistically be performed for the volumes of training data needed in a practical time frame. One or more embodiments generate weak supervision signals for some or all of the training data, allowing training to proceed even when manual ground-truth transcriptions are not available to use as supervisory signals.
3. One or more embodiments further improve the performance of the ASR model by using one or more of (1) data filtering to select high-confidence transcription pairs (audio and transcriptions) as training data, (2) one or more loss functions to preserve knowledge over iterations of the ML model, and/or (3) a multi-layer ML model that supports fine-tuning at different layers.

In an embodiment, improved ASR performance provides faster and more accurate documentation of spoken content (e.g., clinical notes, depositions, etc.), streamlining workflows for workers (e.g., healthcare providers) and reducing administrative burden. Additionally, or alternatively, improved ASR performance provides streamlined data processing. For example, in a medical setting, one or more embodiments may provide real-time transcription of patient encounters, automated entry of electronic health record (“EHR”) data, hands-free interaction with medical systems during procedures, etc. Alternatively, or additionally, improved ASR performance increases documentation speed, reduces transcription costs, and/or improves accessibility for people with disabilities or high workloads. Alternatively, or additionally, improvements in transcription accuracy and domain-specific vocabulary recognition reduce transcription errors with potentially significant benefits. For example, in a medical environment, reducing transcription errors can reduce errors in medical records, provide better compliance with regulatory standards, and/or provide more reliable written data for diagnosis, treatment planning, and/or billing.

### 3. Example ASR Pipeline

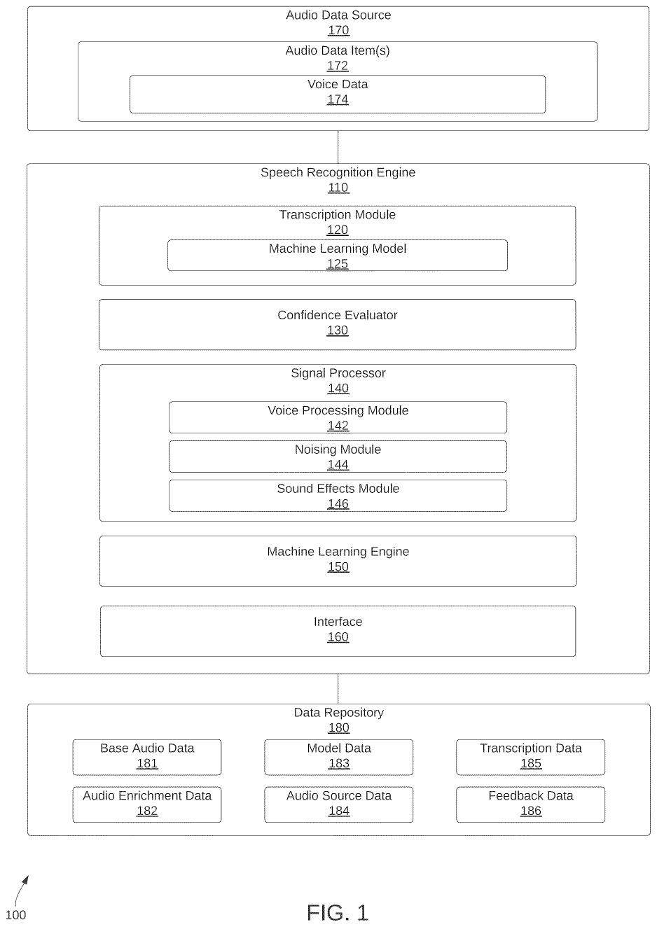

FIG. 1 illustrates an example ASR pipeline 100. In the example, the ASR pipeline 100 includes a speech recognition engine 110, an audio data source 170, and a data repository 180. The speech recognition engine 110 includes a transcription module 120, a confidence evaluator 130, a signal processor 140, a machine learning (ML) engine 150, and an interface 160.

The transcription module 120 is a component that converts audio data into text. In some embodiments, the transcription module 120 includes an ML model 125 that is trained to recognize and transcribe speech. The ML model 125 processes audio signals contained in audio data to generate textual representations of spoken words contained in the audio data. For example, Whisper® (developed by OpenAI®), DeepSpeech® (developed by Mozilla®), Wav2Vec (developed by Meta® Facebook AI®), or Conformer (developed by Google®) perform various speech to text operations such as transcription. In an embodiment, the ML model 125 outputs logits associated with tokens. The logits represent the probabilities of the tokens being correct according to the ML model 125's logic and vocabulary.

The transcription module 120 produces any number of audio-transcription pairs. For example, the ML model 125 generates a first transcription of a first digital recording, a second transcription of a second digital recording, and a third transcription of a third digital recording in the source audio data. For example, the transcription module 120 generates a first audio-transcription pair, a second audio-transcription pair, and a third audio-transcription pair, by pairing the first transcription, the second transcription, and the third transcription with the first digital recording, the second digital recording, and the third digital recording, respectively.

The confidence evaluator 130 refers to a component that estimates a confidence for audio data. For example, the confidence evaluator 130 determines one or more confidence scores for a transcript or other text associated with the audio data. The confidence evaluator determines a confidence score by converting the probabilities associated with logits for tokens output by a model to confidence scores associated with the tokens.

For example, a transcript output by a speech recognition model includes logits that are used by the confidence evaluator 130 to calculate a logit-based confidence score. As used here, a logit is a logarithm of a chance of a particular outcome occurring. In the context of ML models, logits refer to the raw output values that represent a model's confidence in the output. The confidence evaluator 130 uses the logits to estimate a confidence for a transcript by converting the logits to probabilities using a SoftMax function, whereby higher logits indicate greater confidence in predicted tokens. The SoftMax function is a mathematical function that converts a vector of real numbers into a probability distribution over the possible outcomes. The confidence evaluator 130 determines an overall logit-based confidence score for a transcription by averaging probabilities of the tokens included in the transcript.

The confidence evaluator 130 includes a module that compares the transcript to a ground truth transcript for the source audio data to determine an error rate, such as an entity error rate, word error rate, or other error rate, for the transcript. The confidence evaluator 130 determines if a transcript meets a confidence criterion that is based on the error rate and the logit-based confidence. For example, a suitable confidence criterion requires that (1) the error rate is below a threshold, and (2) the logit-based confidence is above a threshold.

The confidence evaluator 130 includes a module that compares a confidence value to one or more thresholds to determine if the confidence value indicates high-confidence, low-confidence, extreme-confidence, or another category. In various embodiments, categories are defined by one or more confidence value thresholds. The thresholds are adjustable depending on requirements for accuracy.

The signal processor 140 includes modules that evaluate, analyze, and/or modify an audio signal included in the audio data. In the example, the signal processor 140 includes a voice processing module 142, a noising module 144, and a sound effects module 146. In various embodiments, the signal processor comprises a suite of various audio editing applications.

The voice processing module 142 introduces augmented voices into audio data to enrich the audio data. The voice processing module 142 includes components that isolate and modify speech signals that are present in audio data. The voice processing module 142 includes filters and/or other signal processing algorithms that isolate voice data and modify it. In some embodiments, voice data in an audio signal is replaced with a modified version of the voice data. For example, the voice processing module 142 replaces a voice in a recording with another voice that speaks the same words with a different accent or intonation.

The noising module 144 introduces random or white noise into audio data to enrich the audio data. The noising module 144 includes various signal processing components that add static or random noise in specified amounts to audio data. In some embodiments, the noising module 144 includes components that add a discrete amount of noise iteratively until a specified criterion is met.

The sound effects module 146 introduces environmental sound effects and other audio elements related to a setting or environment into audio data to enrich the audio data. The sound effects module 146 includes various components that identify environmental sound effects associated with a particular setting. The sound effects module 146 processes audio data to add the environmental sound effects (e.g., medical other environment-specific sound effects) to the audio data. For example, the sound effects are medical sound effects that are recorded in a medical setting and/or downloaded from a medical sound effects database.

The ML engine 150 performs various operations related to ML to train and/or fine-tune the ML model 125. For example, the ML engine 150 is configured to receive enriched audio data and/or a transcript of the enriched audio data and to use the enriched data and/or the transcript as training data during training and/or fine-tuning of the ML model 125 (or another model).

The interface 160 facilitates communicating with external computing devices and/or with input/output operations. The interface 160 refers to hardware and/or software configured to facilitate communication between a user device and a system. In FIG. 1, an interface 160 is used to facilitate communication between the speech recognition engine 110 and other components of the ASR pipeline 100 and/or external components (e.g., one or more user computing devices, client computing devices, and/or administrative computing devices). Such an interface 160 renders user interface elements and receives input via user interface elements.

Examples of interfaces include a graphical user interface (“GUI”), a command line interface (“CLI”), a haptic interface, and a voice command interface. Examples of user interface elements include checkboxes, radio buttons, dropdown lists, list boxes, buttons, toggles, text fields, date and time selectors, command lines, sliders, pages, and forms. In various embodiments, different components of such an interface are specified in different languages. The behavior of user interface elements is specified in a dynamic programming language such as JavaScript. The content of user interface elements is specified in a markup language, such as hypertext markup language (“HTML”) or extensible markup language (“XML”) User Interface Language (“XUL”). The layout of user interface elements is specified in a style sheet language such as Cascading Style Sheets (“CSS”). Alternatively, interfaces may be specified in one or more other languages, such as Java, C, or C++.

In FIG. 1, the audio data source 170 includes one or more audio data items 172. An audio data item 172 includes voice data 174, which consists of a digital representation of speech. The voice data 174 includes one or more voices. For example, in a medical environment, audio data sources may include medical databases, clinical visit recordings, patient discussion recordings, and other dictations.

In one or more embodiments, a data repository 180 is any type of storage unit and/or device (e.g., a file system, database, collection of tables, or any other storage mechanism) for storing data. Further, a data repository 180 may include multiple different storage units and/or devices. The multiple different storage units and/or devices may or may not be of the same type or be located at the same physical site. Further, a data repository 180 may be implemented or executed on the same computing system as other components of the ASR training pipeline 100. Additionally, or alternatively, a data repository 180 may be implemented or executed on a computing system separate from other components of the ASR training pipeline 100. The data repository 180 may be communicatively coupled to one or more other components of the ASR pipeline 100 via a direct connection or via a network.

Information describing base audio data 181, audio enrichment data 182, model data 183, audio source data 184, transcription data 185, and feedback data 186 may be implemented across any of the components within the ASR training pipeline 100. However, this information is illustrated within the data repository 180 for purposes of clarity and explanation.

The base audio data 181 refers to audio data prior to enrichment. The base audio data 181 includes sound recordings in various formats, such as Waveform Audio file format (“.wav”), MPEG-1 Audio Layer III (“.mp3”), Free Lossless Audio Codec (“FLAC”), and/or the like. Base audio data 181 is recorded or loaded from various sources. For example, audio for medical or other professional dictation is captured through handheld digital recorders, mobile dictation applications, or integrated EHR systems.

The audio enrichment data 182 refers to data that is used to enrich other audio data. For example, audio enrichment data 182 includes recorded sound effects, values for parameters for noising algorithms, data related to voice modifications, and the like.

The model data 183 refers to data or metadata related to one or more ML models, including ML model 125. The model data 183 includes usage history, feedback, and/or other model-specific data related to a model trained by and/or deployed by the speech recognition engine 110.

The audio source data 184 includes data or metadata related to the source(s) of the base audio data and/or the audio enrichment data 182. For example, the audio source data 184 identifies the source and other attributes (e.g., type, date of origin, etc.) of the base audio data and the audio enrichment data.

The transcription data 185 refers to textual versions of speech or voice data included in a base audio or enriched audio data item. In embodiments, transcription data 185 includes transcriptions generated by the transcription module 120 and/or ground truth transcriptions received from another source.

The feedback data 186 refers to data indicating a correctness or incorrectness of an output of the ML model 125. In embodiments, the feedback data 186 is used by the ML engine 150 to fine-tune the ML model 125 or to train another ML model such as by performing reinforcement learning.

In an embodiment, the ASR pipeline 100 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware network address translator (“NAT”), a hardware load balancer, a mainframe, a television, a content receiver, a set-top box, a printer, a mobile handset, a smartphone, a personal digital assistant (“PDA”), a wireless receiver and/or transmitter, a base station, a communication management device, a router, a switch, a controller, an access point, and/or a client device.

### 4. Example Operations for Automated Speech Recognition

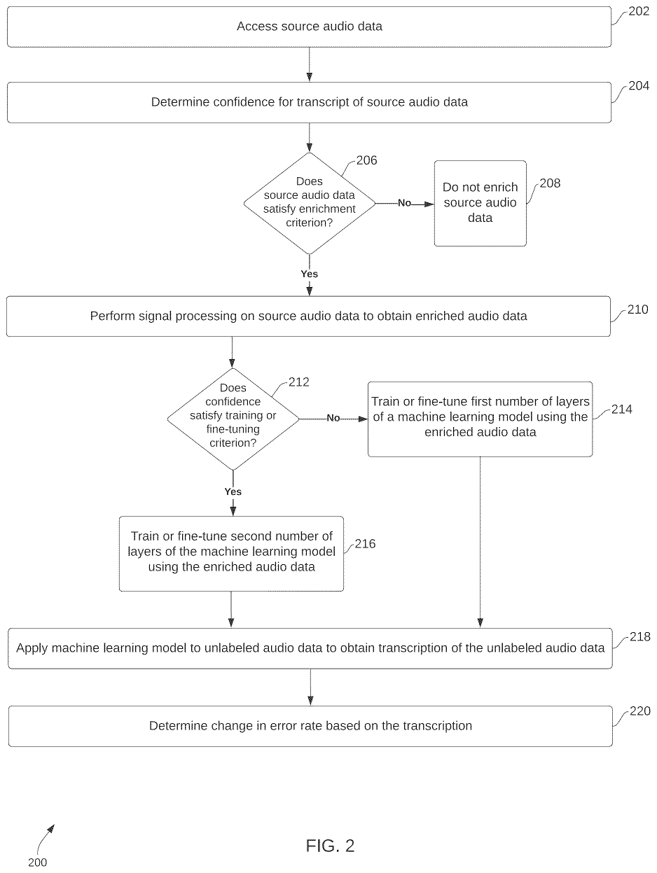

FIG. 2 illustrates an example set of operations 200 for ASR in accordance with one or more embodiments. For example, operations 200 are performable by the speech recognition engine 110 and/or components of the ASR pipeline 100 of FIG. 1.

In FIG. 2, the speech recognition engine accesses source audio data (Operation 202). In general, source audio data may originate from a variety of inputs. Recordings include live meeting recordings, teleconference recordings, audio extracted from video recordings, and the like.

For example, in medical environments, source audio data may originate from dictated clinical notes, surgical recordings, telehealth consultations, etc. Medical source audio data typically includes one or more speakers, such as a doctor, patient, or nurse. Medical source audio data often includes technical terminology, like disease names, drug names, or laboratory names. Medical source audio data also often includes background noise. In some cases, words are pronounced differently by different speakers due to unfamiliarity with a word, an accent, a medical condition, or another reason. However, despite these challenges for speech recognition, accuracy is crucial in many circumstances.

The speech recognition engine determines a confidence for a transcript of source audio data (Operation 204). In this operation, the speech recognition engine generates a transcript from the source audio data by inputting the source audio data into a speech recognition model. The speech recognition model outputs a text corpus corresponding to the speech contained in the source audio data. In some embodiments, the speech recognition engine inputs this text corpus into a large language model (“LLM”) to obtain a healed transcript. The LLM processes the text corpus to correct grammar, spelling, or syntax errors, or to otherwise heal the text corpus without changing the meaning of the text corpus.

Some speech recognition models output logits associated with tokens that represent the words and/or subwords in speech. The speech recognition engine determines a confidence based on logit-based probabilities for the tokens of a transcript and based on an error rate for the transcript. In this example, the speech recognition engine inputs source audio data into a speech recognition model that outputs a transcript of the source audio data. The transcript is made up of tokens used to build words and sub-words that the model determines are present in the source audio data. The speech recognition model outputs logits associated with the tokens. A logit quantifies the likelihood, according to the speech recognition model's logic, that a token predicted by the model is correct. The speech recognition engine determines an overall logit-based confidence for the transcript by averaging or otherwise combining the logit values for the tokens that make up the transcript.

In some embodiments, the speech recognition engine accesses a ground truth transcript of the source audio data. The speech recognition engine compares the transcript that was output by the speech recognition model to the ground truth transcript to determine an error rate (or word error rate) of the transcript output by the model. The speech recognition engine determines if an audio item and associated transcript are high-confidence based on (1) the overall logit-based confidence being above a threshold and (2) the error rate being below another threshold.

The speech recognition engine determines if the source audio data satisfies an enrichment criterion (Operation 206). In this operation, the speech recognition engine compares a confidence and/or error rate for a transcript to one or more thresholds to determine if the transcript is high-confidence (or extreme-confidence).

If the source audio data does not satisfy the enrichment criterion, the speech recognition engine does not enrich the source audio data (Operation 208). For example, the speech recognition engine does not enrich source audio data based on an overall logit-based confidence score for a transcript of the source audio data being too low (i.e., below a threshold). Likewise, the speech recognition engine does not enrich source audio data based on an error rate for a transcript of the source audio data being too high (i.e., above a threshold).

Responsive to the source audio data satisfying the enrichment criterion, the speech recognition engine performs signal processing on the source audio data to obtain enriched audio data (Operation 210). For example, responsive to the overall logit-based confidence for the transcript being above a threshold and the error rate for the transcript being below another threshold, the speech recognition engine selects the source audio data for signal processing.

In embodiments, performing signal processing on the source audio data comprises one or more of the following: applying noise to the source audio data, adding environmental sound effects to the source audio data, or modifying a speaking voice included in the source audio data.

Applying noise to the source audio data includes adding Gaussian or other types of noise. In embodiments, the speech recognition engine applies noise in quantified amounts by controlling the magnitude of a noise vector. In this example, the statistical properties of the noise vector define the type of noise that is being applied, and the magnitude of the noise vector defines the amount of noise.

Adding environmental sounds to the source audio includes merging various sound effects into the source audio to generate enriched audio. Some environmental sounds are recorded from a type of environment that matches a target deployment setting for a target model trained using the enriched audio. Some environmental sounds are identified based on being associated with the type of environment and loaded from a database or another source.

Modifying a speaking voice included in the source audio includes identifying one or more speaking voices, extracting the portion of the source audio data associated with a particular speaking voice, modifying the particular speaking voice, and/or merging the modified speaking voice into the source audio, with the original particular speaking voice removed, to obtain enriched audio data. In various embodiments, the speech recognition engine modifies the voice by changing an accent, intonation, or volume of the voice, and/or by otherwise manipulating the voice.

The speech recognition engine determines whether the confidence satisfies a training or fine-tuning criterion (Operation 212). In this operation, the speech recognition engine compares a confidence score, such as an overall logit-based confidence score for a transcript, to a training or fine-tuning criterion to determine if the transcript is extreme-confidence. In this example, extreme-confidence is higher-confidence than high-confidence.

If the speech recognition engine determines that the confidence does not satisfy the training or fine-tuning criterion, the speech recognition engine trains or fine-tunes a first number of layers of a ML model using the enriched audio data (Operation 214). In this example, the speech recognition engine trains less than all of the layers of the ML model based on the confidence being below a threshold for determining extreme-confidence.

Responsive to the speech recognition engine determining that the confidence does satisfy the training or fine-tuning criterion, the speech recognition engine trains or fine-tunes a second number of layers of the ML model using the enriched audio data (Operation 216). In this operation, the speech recognition engine trains a second number of layers of the ML model based on the confidence being above a threshold for determining extreme-confidence. For example, an ML model has n layers. In response to the speech recognition engine determining that the confidence satisfies an extreme-confidence criterion, the speech recognition engine trains and/or fine-tunes n layers of the ML model.

The speech recognition engine applies the ML model to unlabeled audio data to obtain a transcription of the unlabeled audio data (Operation 218). In this operation, the speech recognition engine receives or otherwise accesses unlabeled audio data. The speech recognition engine inputs the unlabeled audio data into the ML model to cause the ML model to output a transcription of the input unlabeled audio data. The unlabeled audio data refers to a stored recording, live dictation, or other audio.

The speech recognition engine determines a change in error rate based on the transcription (Operation 220). In this operation, the speech recognition engine compares an error rate of the transcription output by the ML model to a previous error rate for another version of the ML model before training. In embodiments, the speech recognition engine accesses additional audio data, enriches the additional audio data to obtain additional enriched audio data, and trains and/or fine-tunes the ML model with the additional enriched audio data. The speech recognition engine determines one or more subsequent error rates and determines a rate of change of the error rate based on the previous error rate, the error rate, and/or the one or more subsequent error rates. Various error rates include a match error rate, entity error rate, word error rate, character error rate, or the like.

### 5. Examples of ASR

### 5.1. Source Audio Data Transcription

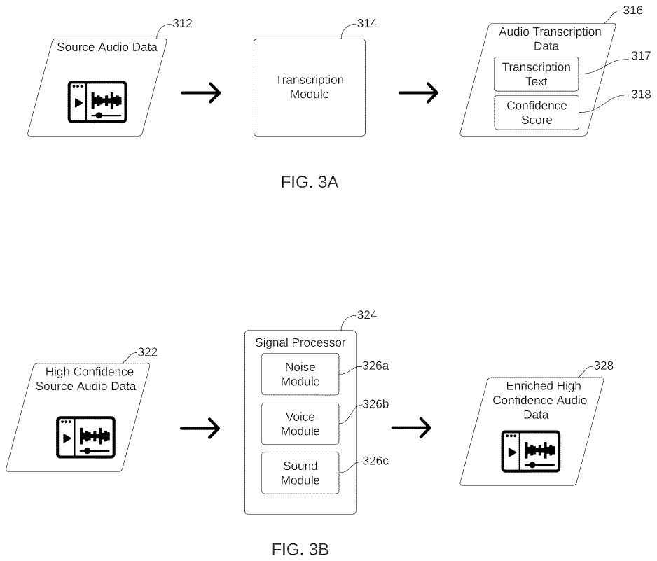

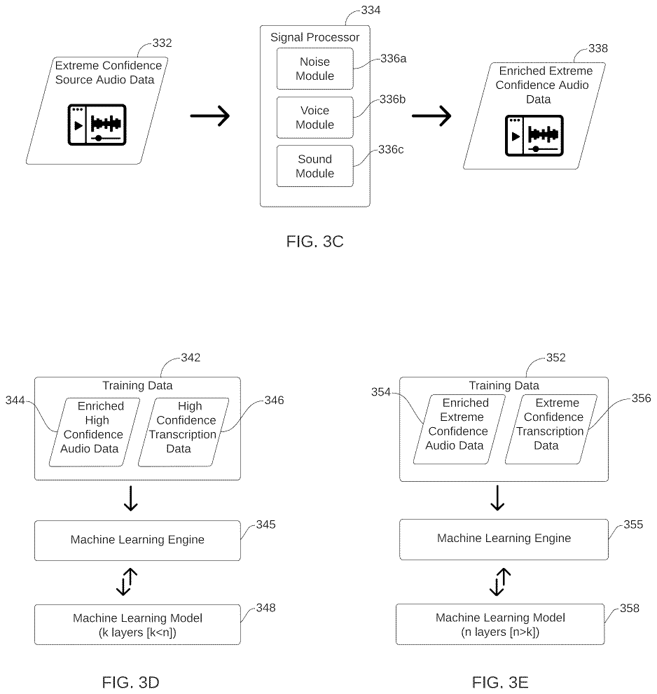

FIG. 3A illustrates an example of a speech recognition pipeline processing source audio data 312 to obtain audio transcription data 316. In FIG. 3A, source audio data 312 is input into a transcription module 314 to result in the transcription module 314 outputting the audio transcription data 316. For example, the pipeline interfaces with a speech recognition model to obtain an initial transcript of the source audio data 312. Various suitable models include private models as well as base versions or tuned versions of models such as Whisper® (developed by OpenAI®), DeepSpeech® (developed by Mozilla®), Wav2Vec (developed by Meta® Facebook AI®), Conformer (developed by Google®), and/or the like.

In an embodiment, the output audio transcription data 316 includes a transcription text 317 and a confidence score 318 for the transcription text 317. The transcription module 314 performs speech recognition on the source audio data 312 by using a speech recognition model to generate the transcription text 317 and the confidence score 318. In some embodiments, the transcription text 317 comprises a set of tokens selected based on having a highest likelihood, among? tokens in a vocabulary, of being valid or correct.

In the example, the transcription text 317 includes words, phrases, or utterances recognized in the source audio data by the transcription module 314. The speech recognition pipeline computes the confidence score 318 by averaging the result of applying a SoftMax function to the logits associated with the tokens in the output from the speech recognition model. In this context, a logit quantifies a likelihood that the speech recognition model considers a token to be valid or correct. Transcripts for which the tokens have a high likelihood of being valid have a high confidence score.

The speech recognition pipeline compares confidence scores to threshold values to determine if audio data is high-confidence or extreme-confidence audio data. The threshold values are adjustable based on the quality of the source audio data, the amount of the source audio data, a base performance of a model, an adapted performance of the model, and/or a target training time. For example, the speech recognition pipeline selects a relatively higher threshold value based on a high availability of data or a high quality of data. The pipeline selects a relatively higher extreme confidence threshold based on a strong base performance. The pipeline selects a relatively lower high confidence threshold but not a relatively lower extreme confidence threshold based on strong base performance and poor adapted performance. In various embodiments, the pipeline utilizes one or more additional threshold values to categorize data by confidence into one or more additional categories.

### 5.2. High Confidence Audio Data Enrichment

FIG. 3B illustrates an example of processing high confidence source audio data 322 to obtain enriched high confidence audio data 328. In FIG. 3B, high confidence source audio data 322 is input into a signal processor 324.

The signal processor 324 refers to a suite of applications or functions that manipulate audio data to add or modify sounds, noise, or voices, such as by injecting synthetic audio elements, modifying existing voice recordings, masking, changing the speed of a recording, or the like. In the example, the signal processor 324 includes a noise module 326a, a voice module 326b, and a sound module 326c.

The noise module 326a includes filters and generation components configured to apply or overlay noise patterns onto audio data. This includes Gaussian noise, white noise, or random background noise. The noise module 326a is composed of signal generators, noise profiling tools, and frequency band adjusters to control the acoustic characteristics of the added noise. For example, the noise module 326a adds an amount of noise to audio data corresponding to the magnitude of a noise vector defined by the noise module 326a. In an embodiment, the noise module 326a generates multiple audio data items from a source audio by adding different amounts of noise to the source audio in a stepwise manner by increasing the magnitude of the noise vector applied to the source audio.

The voice module 326b includes submodules related to voice characteristics in audio data. These include components for altering pitch, tone, accent, cadence, etc. The voice module 326b includes waveform manipulators, phoneme-level transformation engines, and the like. For example, the voice module 326b replaces a voice in audio data with a modified voice. In embodiments, the voice module 326b produces any number of new audio data items by replacing a voice with a different synthetic voice.

The sound module 326c includes components that manage sound effects or background sounds that are specific to a particular setting and that overlay or inject the sounds into audio data. For example, the sound module 326c layers one or more environmental sounds from a sound database onto audio data to enrich the audio data. Example medical environmental sounds include a coughing sound, a blood pressure machine sound, a vitals monitor sound, another medical device sound, or the like. In another example, environmental sounds for a speaker that is in a car include car horns, car doors, traffic sounds, windshield wipers' sounds, other car sounds, etc. In an embodiment, the sound module 326c adds one or more sounds to audio data corresponding to a deployment environment to obtain the enriched audio. The enriched audio data is used to train a speech recognition model for the deployment environment.

The enriched high confidence audio data 328 includes the same spoken words as the high confidence source audio data 322 except that the enriched high confidence audio data 328 has undergone signal processing, so the enriched audio includes added noise, a modified voice, and/or an added environmental sound as compared to the high confidence source audio data 322 before enrichment. Although the audio is modified, the transcript for the high confidence source audio data 322 aligns with the enriched high confidence audio data 328. The transcript and the enriched high confidence audio data 328 are used to train and/or fine tune a speech recognition model using an elastic weight consolidation-based loss function.

### 5.3. Extreme Confidence Audio Data Enrichment

FIG. 3C illustrates an example of processing extreme confidence source audio data 332 to obtain enriched extreme confidence audio data 338. In FIG. 3C, extreme confidence source audio data 332 is input into a signal processor 334. The extreme confidence source audio data 332 is paired with a transcription with an extremely high confidence. In various embodiments, a threshold for determining extreme confidence is selected based on a confidence score threshold (e.g., a confidence within a threshold distance from 1.00) or a confidence percentile (e.g., a top ranking percentile for a confidence score for a set of audios).

The signal processor 334 is similar to the signal processor 324 and also includes a suite of applications or functions that manipulate audio data to add or modify sounds, including a noise module 336a, a voice module 336b, and a sound module 336c. In the example, the noise module 336a, voice module 336b, and sound module 336c perform operations and include components similar to noise module 326a, voice module 326b, and sound module 326c.

In this example, the enriched extreme confidence audio data 338 is similar to the extreme confidence source audio data 332 except that the enriched extreme confidence audio data 338 has undergone signal processing, so the enriched extreme confidence audio data 338 includes added noise, a modified voice, and/or an added environmental sound, as compared to the extreme confidence source audio data 332 before enrichment.

### 5.4. Machine Learning Using Enriched High Confidence Audio Data

FIG. 3D illustrates an example of machine learning using enriched high confidence audio data. In FIG. 3D, training data 342 comprises enriched high confidence audio data 344 and high confidence transcription data 346. In this example, the high confidence transcription data 346 comprises a transcription of high confidence audio data used to obtain the enriched high confidence audio data 344.

In the example, a speech recognition system inputs the training data 342 into an ML engine 345 to cause the ML engine 345 to train k layers of a ML model 348. In this example, the ML model 348 includes n layers, and the number of layers, k, that the engine trains and/or fine-tunes is less than n.

In some embodiments, the ML engine 345 trains a number of layers of the ML model 348 that is randomly selected from between 1 and k, inclusive. For example, for a high-confidence audio item, the system generates a random number r between 1 and k and adjusts parameters of the ML model associated with the top r layers of the ML model. In this context, the bottom layers are considered foundational or base layers, whereas the top layers are considered adaptive or adaptation layers. In some embodiments, k is selected so the adaptive layers of the model are tuned without one or more foundational layers being included in the tuning.

### 5.5. Machine Learning Using Enriched Extreme Confidence Audio Data

FIG. 3E illustrates an example of machine learning using enriched extreme confidence audio data. In FIG. 3E, training data 352 comprises enriched extreme confidence audio data 354 and extreme confidence transcription data 356. In this example, the extreme confidence transcription data 356 comprises a transcription of extreme confidence audio data used to obtain the enriched extreme confidence audio data 354.

In the example, a speech recognition system inputs the training data 352 into an ML engine 355 to cause the ML engine 355 to train n layers of a ML model 358. In this example, the ML model 358 includes n layers.

In embodiments, one or more audio data items satisfy a high confidence threshold criterion but not an extreme confidence threshold criterion. An ML engine uses audio data items that satisfy the high confidence threshold criterion but not the extreme confidence threshold criterion to train k layers of the ML model. The ML engine uses audio data items that satisfy the extreme confidence threshold criterion to train n layers of the ML model, where n is greater than k. In various embodiments, one or more additional thresholds are used to determine the number of layers trained by the ML engine.

### 5.6. Elastic Weight Consolidation-Based Loss Function

Performing machine learning using an elastic weight consolidation (“EWC”) based loss function facilitates ML from new data without losing knowledge of previously learned tasks. This technique helps the model to retain prior knowledge while adapting to new tasks or data. The EWC loss function adds a penalty term to a loss function, which constrains changes to certain weights such as those for previously learned tasks. This penalty is determined using a Fisher Information Matrix, which identifies parameters that are not significantly altered during fine-tuning.

In embodiments, the EWC loss function for an ML model includes a weighting mechanism. A training sample is assigned a weight proportional to its transcription confidence score. Samples with higher confidence scores exert a stronger influence on the loss function. This causes the model to have a greater response and prioritize learning from the data that is considered highly reliable based on confidence score. An example EWC loss function is defined as

L=L_{CE}+λC*Sum(Fᵢ(θᵢ−θ*ᵢ)²),
where L_{CE} is the cross-entropy loss. The index i refers to a number of individual parameters of the ML model. θᵢ represents the current model parameters. θ*ᵢ denotes the optimal parameters from the previous model. Fᵢ corresponds to the diagonal values of the Fisher Information Matrix, representing how much information a parameter carries about the data and indicating its importance. Sum(Fᵢ(θᵢ−θ*ᵢ)²) is the sum of Fᵢ(θᵢ−θ*ᵢ)² for the i parameters of the model. C is the transcription confidence, and λ is the regularization strength. A higher C value reflects more accurate transcriptions by the previous model, resulting in a higher penalty for deviations in the current model. In an embodiment, the regularization strength hyperparameter λ is determined experimentally by attempting all values in the test range of [0.1, 0.3, 0.5, 0.8, 1, 2, 3, 4, 5, 6, 7, 8, 9, 10, 20, 30]. Based on experimentation, λ=1 gave good performance although other values in the test range (or outside the test range) are usable.

This adaptive weighting approach enhances the overall robustness and precision of the model where accuracy is critical. The combination of EWC and confidence-based weighting results in training for the model that entirely or substantially retains the model's prior knowledge while incorporating new data.

### 6. Machine Learning Architecture

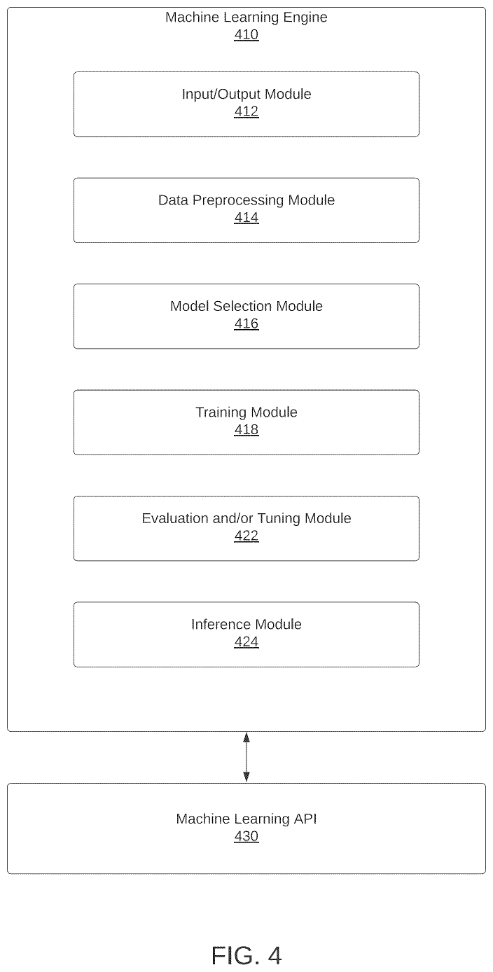

FIG. 4 illustrates a machine learning engine 410 in accordance with one or more embodiments. As illustrated in FIG. 4, ML engine 410 includes input/output module 412, data preprocessing module 414, model selection module 416, training module 418, evaluation and/or tuning module 422, and inference module 424.

In accordance with an embodiment, input/output module 412 serves as the primary interface for data entering and exiting the system, managing the flow and integrity of data. This module may accommodate a wide range of data sources and formats to facilitate integration and communication within the ML architecture.

In an embodiment, an input handler within input/output module 412 includes a data ingestion framework capable of interfacing with various data sources, such as databases, Application Programming Interfaces (“API”)s, file systems, and real-time data streams. This framework is equipped with functionalities to handle different data formats (e.g., CSV, JSON, XML) and efficiently manage large volumes of data. It includes mechanisms for batch and real-time data processing that enable the input/output module 412 to be versatile in different operational contexts whether processing historical datasets or streaming data.

In accordance with an embodiment, input/output module 412 manages data integrity and quality as it enters the system by incorporating initial checks and validations. These checks and validations ensure that incoming data meets predefined quality standards, like checking for missing values, ensuring consistency in data formats, and verifying data ranges and types. This proactive approach to data quality minimizes potential errors and inconsistencies in later stages of the ML process.

In an embodiment, an output handler within input/output module 412 includes an output framework designed to handle the distribution and exportation of outputs, predictions, or insights. Using the output framework, input/output module 412 formats these outputs into user-friendly and accessible formats, such as reports, visualizations, or data files, compatible with other systems. Input/output module 412 also ensures secure and efficient transmission of these outputs to end-users or other systems in an embodiment and may employ encryption and secure data transfer protocols to maintain data confidentiality.

In accordance with an embodiment, data preprocessing module 414 transforms data into a format suitable for use by other modules in ML engine 410. For example, data preprocessing module 414 may transform raw data into a normalized or standardized format suitable for training ML models and for processing new data inputs for inference. In an embodiment, data preprocessing module 414 acts as a bridge between the raw data sources and the analytical capabilities of ML engine 410.

In an embodiment, data preprocessing module 414 begins by implementing a series of preprocessing steps to clean, normalize, and/or standardize the data. This involves handling a variety of anomalies, such as managing unexpected data elements, recognizing inconsistencies, or dealing with missing values. Some of these anomalies can be addressed through methods, like imputation or removal of incomplete records, depending on the nature and volume of the missing data. Data preprocessing module 414 may be configured to handle anomalies in different ways, depending on context. Data preprocessing module 414 also handles the normalization of numerical data in preparation for use with models sensitive to the scale of the data, like neural networks and distance-based algorithms. Normalization techniques, such as min-max scaling or z-score standardization, may be applied to bring numerical features to a common scale, enhancing the model's ability to learn effectively.

In an embodiment, data preprocessing module 414 includes a feature encoding framework that ensures categorical variables are transformed into a format that can be easily interpreted by ML algorithms. Techniques, such as one-hot encoding or label encoding, may be employed to convert categorical data into numerical values, making them suitable for analysis. The module may also include feature selection mechanisms, where redundant or irrelevant features are identified and removed, thereby increasing the efficiency and performance of the model.

In accordance with an embodiment, when data preprocessing module 414 processes new data for inference, data preprocessing module 414 replicates the same preprocessing steps to ensure consistency with the training data format. This helps to avoid discrepancies between the training data format and the inference data format, thereby reducing the likelihood of inaccurate or invalid model predictions.

In an embodiment, model selection module 416 includes logic for determining the most suitable algorithm or model architecture for a given dataset and problem. This module operates in part by analyzing the characteristics of the input data, such as its dimensionality, distribution, and the type of problem (classification, regression, clustering, etc.).

In an embodiment, model selection module 416 employs a variety of statistical and analytical techniques to understand data patterns, identify potential correlations, and assess the complexity of the task. Based on this analysis, it then matches the data characteristics with the strengths and weaknesses of various available models. This can range from simple linear models for less complex problems to sophisticated deep learning architectures for tasks requiring feature extraction and high-level pattern recognition, such as image and speech recognition.

In an embodiment, model selection module 416 utilizes techniques from the field of Automated Machine Learning (“AutoML”). AutoML systems automate the process of model selection by rapidly prototyping and evaluating multiple models. They use various techniques, like Bayesian optimization, genetic algorithms, or reinforcement learning, to explore the model space efficiently. Model selection module 416 may use these techniques to evaluate each candidate model based on performance metrics relevant to the task. For example, accuracy, precision, recall, or F1 score may be used for classification tasks, and mean squared error metrics may be used for regression tasks. Accuracy measures the proportion of correct predictions (both positive and negative). Precision measures the proportion of actual positives among the predicted positive cases. Recall (also known as sensitivity) evaluates how well the model identifies actual positives. F1 Score is a single metric that accounts for both false positives and false negatives. The mean squared error (“MSE”) metric may be used for regression tasks. Mean squared error measures the average squared difference between the actual and predicted values, providing an indication of the model's accuracy. A lower MSE may indicate a model's greater accuracy in predicting values, for it represents a smaller average discrepancy between the actual and predicted values.

In accordance with an embodiment, model selection module 416 also considers computational efficiency and resource constraints. This is meant to help ensure the selected model is both accurate and practical in terms of computational and time requirements. In an embodiment, certain features of model selection module 416 are configurable such as a configured bias toward (or against) computational efficiency.

In accordance with an embodiment, training module 418 manages the ‘learning’ process of ML models by implementing various learning algorithms that enable models to identify patterns and make predictions or decisions based on input data. In an embodiment, the training process begins with the preparation of the dataset after preprocessing; this involves splitting the data into training and validation sets. The training set is used to teach the model, while the validation set is used to evaluate its performance and adjust parameters accordingly. Training module 418 handles the iterative process of feeding the training data into the model, adjusting the model's internal parameters (like weights in neural networks) through backpropagation and optimization algorithms, such as stochastic gradient descent or other algorithms providing similarly useful results.

In accordance with an embodiment, training module 418 manages overfitting, where a model learns the training data too well, including its noise and outliers, at the expense of its ability to generalize to new data. Techniques, such as regularization, dropout (in neural networks), and early stopping, are implemented to mitigate this. Additionally, the module employs various techniques for hyperparameter tuning; this involves adjusting model parameters that are not directly learned from the training process, such as learning rate, the number of layers in a neural network, or the number of trees in a random forest.

In an embodiment, training module 418 includes logic to handle different types of data and learning tasks. For instance, it includes different training routines for supervised learning (where the training data comes with labels) and unsupervised learning (without labeled data). In the case of deep learning models, training module 418 also manages the complexities of training neural networks that include initializing network weights, choosing activation functions, and setting up neural network layers.

In an embodiment, evaluation and/or tuning module 422 incorporates dynamic feedback mechanisms and facilitates continuous model evolution to help ensure the system's relevance and accuracy as the data landscape changes. Evaluation and/or tuning module 422 conducts a detailed evaluation of a model's performance. This process involves using statistical methods and a variety of performance metrics to analyze the model's predictions against a validation dataset. The validation dataset, distinct from the training set, is instrumental in assessing the model's predictive accuracy and its capacity to generalize beyond the training data. The module's algorithms meticulously dissect the model's output, uncovering biases, variances, and the overall effectiveness of the model in capturing the underlying patterns of the data.

In an embodiment, evaluation and/or tuning module 422 performs continuous model tuning by using hyperparameter optimization. Evaluation and/or tuning module 422 performs an exploration of the hyperparameter space using algorithms, such as grid search, random search, or more sophisticated methods like Bayesian optimization. Evaluation and/or tuning module 422 uses these algorithms to iteratively adjust and refine the model's hyperparameters-settings that govern the model's learning process but are not directly learned from the data-to enhance the model's performance. This tuning process helps to balance the model's complexity with its ability to generalize and attempts to avoid the pitfalls of underfitting or overfitting.

In an embodiment, evaluation and/or tuning module 422 integrates data feedback and updates the model. Evaluation and/or tuning module 422 actively collects feedback from the model's real-world applications, an indicator of the model's performance in practical scenarios. Such feedback can come from various sources, depending on the nature of the application. For example, in a user-centric application, like a recommendation system, feedback might comprise user interactions, preferences, and responses. In other contexts, such as predicting events, it might involve analyzing the model's prediction errors, misclassifications, or other performance metrics in live environments.

In an embodiment, feedback integration logic within evaluation and/or tuning module 422 integrates this feedback using a process of assimilating new data patterns, user interactions, and error trends into the system's knowledge base. The feedback integration logic uses this information to identify shifts in data trends or emergent patterns that were not present or inadequately represented in the original training dataset. Based on this analysis, the module triggers a retraining or updating cycle for the model. If the feedback suggests minor deviations or incremental changes in data patterns, the feedback integration logic may employ incremental learning strategies, fine-tuning the model with the new data while retaining its previously learned knowledge. In cases where the feedback indicates significant shifts or the emergence of new patterns, a more comprehensive model updating process may be initiated. This process might involve revisiting the model selection process, re-evaluating the suitability of the current model architecture, and/or potentially exploring alternative models or configurations that are more attuned to the new data.

In accordance with an embodiment, throughout this iterative process of feedback integration and model updating, evaluation and/or tuning module 422 employs version control mechanisms to track changes, modifications, and the evolution of the model, facilitating transparency and allowing for rollback if necessary. This continuous learning and adaptation cycle, driven by real-world data and feedback, helps to endure the model's ongoing effectiveness, relevance, and accuracy.

In an embodiment, inference module 424 transforms raw data into actionable, precise, and contextually relevant predictions. In addition to processing and applying a trained model to new data, inference module 424 may also include post-processing logic that refines the raw outputs of the model into meaningful insights.

In an embodiment, inference module 424 includes classification logic that takes the probabilistic outputs of the model and converts them into definitive class labels. This process involves an analytical interpretation of the probability distribution for each class. For example, in binary classification, the classification logic may identify the class with a probability above a certain threshold, but classification logic may also consider the relative probability distribution between classes to create a more nuanced and accurate classification.

In an embodiment, inference module 424 transforms the outputs of a trained model into definitive classifications. Inference module 424 employs the underlying model as a tool to generate probabilistic outputs for each potential class. It then engages in an interpretative process to convert these probabilities into concrete class labels.

In an embodiment, when inference module 424 receives the probabilistic outputs from the model, it analyzes these probabilities to determine how they are distributed across some or every potential class. If the highest probability is not significantly greater than the others, inference module 424 may determine that there is ambiguity or interpret this as a lack of confidence displayed by the model.

In an embodiment, inference module 424 uses thresholding techniques for applications where making a definitive decision based on the highest probability might not suffice due to the critical nature of the decision. In such cases, inference module 424 assesses if the highest probability surpasses a certain confidence threshold that is predetermined based on the specific requirements of the application. If the probabilities do not meet this threshold, inference module 424 may flag the result as uncertain or defer the decision to a human expert. Inference module 424 dynamically adjusts the decision thresholds based on the sensitivity and specificity requirements of the application, subject to calibration for balancing the trade-offs between false positives and false negatives.

In accordance with an embodiment, inference module 424 contextualizes the probability distribution against the backdrop of the specific application. This involves a comparative analysis, especially in instances where multiple classes have similar probability scores, to deduce the most plausible classification. In an embodiment, inference module 424 may incorporate additional decision-making rules or contextual information to guide this analysis, ensuring that the classification aligns with the practical and contextual nuances of the application.

In regression models, where the outputs are continuous values, inference module 424 may engage in a detailed scaling process in an embodiment. Outputs, often normalized or standardized during training for optimal model performance, are rescaled back to their original range. This rescaling involves recalibration of the output values using the original data's statistical parameters, such as mean and standard deviation, ensuring that the predictions are meaningful and comparable to the real-world scales they represent.

In an embodiment, inference module 424 incorporates domain-specific adjustments into its post-processing routine. This involves tailoring the model's output to align with specific industry knowledge or contextual information. For example, in financial forecasting, inference module 424 may adjust predictions based on current market trends, economic indicators, or recent significant events, ensuring that the outputs are both statistically accurate and practically relevant.

In an embodiment, inference module 424 includes logic to handle uncertainty and ambiguity in the model's predictions. In cases where inference module 424 outputs a measure of uncertainty, such as in Bayesian inference models, inference module 424 interprets these uncertainty measures by converting probabilistic distributions or confidence intervals into a format that can be easily understood and acted upon. This provides users with both a prediction and an insight into the confidence level of that prediction. In an embodiment, inference module 424 includes mechanisms for involving human oversight or integrating the instance into a feedback loop for subsequent analysis and model refinement.

In an embodiment, inference module 424 formats the final predictions for end-user consumption. Predictions are converted into visualizations, user-friendly reports, or interactive interfaces. In some systems, like recommendation engines, inference module 424 also integrates feedback mechanisms, where user responses to the predictions are used to continually refine and improve the model, creating a dynamic, self-improving system.

The machine learning engine API 430 is an interface that facilitates access to and interaction with the ML engine 410 by other modules and/or components of a system. In an embodiment, ML engine API 430 allows for applications to leverage ML engine 410. In an embodiment, ML engine API 430 may be built on a RESTful architecture and offer stateless interactions over standard HTTP/HTTPS protocols. Machine learning engine API 430 may feature a variety of endpoints, each tailored to a specific function within ML engine 410. In an embodiment, endpoints such as “/submitData” facilitate the submission of new data for processing, while endpoints such as “/retrieveResults” fetch the outcomes of data analysis or model predictions. Message level encryption (“MLE”) API also includes endpoints, such as “/updateModel” for model modifications and “/trainModel” to initiate training with new datasets.

In an embodiment, ML engine API 430 is equipped to support SOAP-based interactions. This extension involves defining a Web Services Description Language (“WSDL”) document that outlines the API's operations and the structure of request and response messages. In an embodiment, ML engine API 430 supports various data formats and communication styles. In an embodiment, ML engine API 430 endpoints may handle requests in JSON format or any other suitable format. For example, ML engine API 430 may process XML, and it may also be engineered to handle more compact and efficient data formats, such as Protocol Buffers or Avro, for use in bandwidth-limited scenarios.

In an embodiment, ML engine API 430 is designed to integrate WebSocket technology for applications necessitating real-time data processing and immediate feedback. This integration enables a continuous, bi-directional communication channel for a dynamic and interactive data exchange between the application and ML engine 410.

### 7. Machine Learning Operations

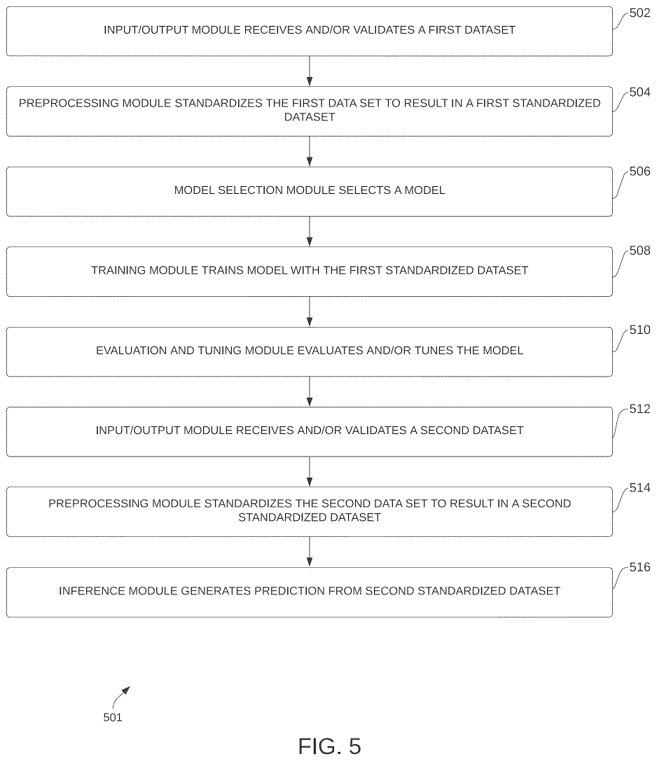

FIG. 5 illustrates a set of machine learning operations 501. In embodiments, one or more operations of the set of operations 500 is performed by an ML engine such as ML engine 410. In an embodiment, input/output module 412 receives a dataset intended for training (Operation 502). This data can originate from diverse sources, like databases or real-time data streams, and in varied formats, such as CSV, JSON, or XML. Input/output module 412 assesses and validates the data, ensuring its integrity by checking for consistency, data ranges, and types.

In an embodiment, training data is passed to data preprocessing module 414. Here, the data undergoes a series of transformations to standardize and clean it, making it suitable for training ML models (Operation 504). This involves normalizing numerical data, encoding categorical variables, and handling missing values through techniques like imputation.

In an embodiment, prepared data from the data preprocessing module 414 is then fed into model selection module 416 (Operation 506). This module analyzes the characteristics of the processed data, such as dimensionality and distribution, and selects the most appropriate model architecture for the given dataset and problem. It employs statistical and analytical techniques to match the data with an optimal model, ranging from simpler models for less complex tasks to more advanced architectures for intricate tasks.

In an embodiment, training module 418 trains the selected model with the prepared dataset (Operation 508). It implements learning algorithms to adjust the model's internal parameters, optimizing them to identify patterns and relationships in the training data. Training module 418 also addresses the challenge of overfitting by implementing techniques, like regularization and early stopping, ensuring the model's generalizability.

In an embodiment, evaluation and/or tuning module 422 evaluates the trained model's performance using the validation dataset (Operation 510). Evaluation and/or tuning module 422 applies various metrics to assess predictive accuracy and generalization capabilities. It then tunes the model by adjusting hyperparameters, and if needed, incorporates feedback from the model's initial deployments, retraining the model with new data patterns identified from the feedback.

In an embodiment, input/output module 412 receives a dataset intended for inference. Input/output module 412 assesses and validates the data (Operation 512).

In an embodiment, data preprocessing module 414 receives the validated dataset intended for inference (Operation 514). Data preprocessing module 414 ensures that the data format used in training is replicated for the new inference data, maintaining consistency and accuracy for the model's predictions.

In an embodiment, inference module 424 processes the new data set intended for inference, using the trained and tuned model (Operation 516). It applies the model to this data, generating raw probabilistic outputs for predictions. Inference module 424 then executes a series of post-processing steps on these outputs, such as converting probabilities to class labels in classification tasks or rescaling values in regression tasks. It contextualizes the outputs as per the application's requirements, handling any uncertainty in predictions and formatting the final outputs for end-user consumption or integration into larger systems.

### 8. Generative Artificial Intelligence Models

A generative model is an ML model that is capable of generating new data instances based on the data used to train the model. A generative model may be referred to as a “generative artificial intelligence (AI) model.” Generative models learn the underlying distribution of the training data, enabling them to produce new instances of data that share properties with the original dataset. This capability makes them particularly useful in a variety of applications, including image and voice generation, text synthesis, and more sophisticated tasks, such as unsupervised learning, semi-supervised learning, and domain adaptation.

Large language models are designed to understand, generate, and interpret human language by processing extensive collections of data. The foundational architecture behind LLMs is the transformer network, a type of neural network that excels in handling sequential data such as text. Unlike certain architectures, such as recurrent neural networks (“RNNs”) or long short-term memory networks (“LSTMs”), transformers do not process data in order. Instead, they leverage parallel processing to analyze entire text sequences simultaneously, significantly improving efficiency and reducing training times.

In an embodiment, a mechanism that enables transformers to handle complex language tasks is self-attention. This mechanism allows the model to weigh the importance of different words within a sentence or sequence regardless of their position. For instance, in processing the phrase “The cat sat on the mat,” the model can directly associate “cat” with “mat” without having to process the intermediate words sequentially. This ability to understand the context and relationships between words in a sentence is what makes transformer networks adept at language tasks. The self-attention mechanism assigns scores to relationships between words, highlighting the most relevant connections, so the model can focus on the most informative parts of the text.

In accordance with one or more embodiments, transformers are composed of multiple layers including a multi-head, self-attention mechanism and a position-wise, feed-forward network. Within the architecture of transformer models, the multi-head, self-attention mechanism and position-wise, feed-forward network function in concert to process input data. The multi-head, self-attention mechanism is designed to enable parallel processing of input sequences, allowing the model to simultaneously evaluate the importance of different segments of the input relative to each other. This mechanism operates by generating multiple sets of query, key, and value vectors for each element in the input sequence through linear transformation. The relevance of each element to other elements is calculated using a scaled dot-product attention function that computes the attention scores by taking the dot product of the query vector with the key vectors, dividing each by the square root of the dimension of the key vectors to scale the scores, then applying a “SoftMax” function to obtain the weights for the value vectors. The scaled dot-product attention function is applied independently by each head in the multi-head, self-attention mechanism. The outputs of these heads are then concatenated and linearly transformed, allowing the model to capture information from different representation subspaces.

In accordance with one or more embodiments, following the multi-head, self-attention mechanism is the position-wise, feed-forward network. This component comprises two linear transformations with a non-linear activation function in between. Each element of the input sequence, now enriched with context by the self-attention mechanism, is processed independently through the same feed-forward network. The first linear transformation increases the dimensionality of the input, allowing for a richer representation space. The non-linear activation function introduces the capability to capture non-linear relationships within the data. The second linear transformation then reduces the dimensionality back to that of the model's hidden layers, preparing the output for either further processing by subsequent layers or final output generation. This sequence of operations is applied to each position in the sequence, so the model can learn complex patterns across different parts of the input data without relying on the sequential processing inherent to previous architectures, such as RNNs or LSTMs.

In accordance with one or more embodiments, integrating these components within the transformer architecture facilitates the model's ability to understand and generate human language by leveraging both the global context provided by the self-attention mechanism and the local, position-specific transformations applied by the feed-forward networks. Through the repetitive stacking of layers, transformers achieve a depth of representation that allows for the processing of linguistic information across varying levels of complexity.

In accordance with one or more embodiments, input/output module 412, when used for LLMs, handles textual data, converting input text into a format that the model can process. This typically involves tokenization, where the text is broken down into manageable pieces, such as words or subwords, and then converted into numerical representations. These representations, or embeddings, capture semantic information about the text that is then fed into the model for processing. The output from the model is converted from numerical form back into human-readable text, following the generation of predictions or responses.

In accordance with one or more embodiments, data preprocessing module 414 in the context of LLMs may include steps, such as normalization, where the text is converted to a uniform case, and punctuation is standardized. This process ensures that the model treats similar words or symbols consistently, reducing the complexity of the input space. Additionally, techniques, such as sentence segmentation, may be applied to manage longer texts, enabling the model to process information in chunks that align with natural language structures.

In accordance with one or more embodiments, model selection module 416, when used for LLMs, involves choosing a specific architecture and configuration that is best suited to the task at hand. This decision is based on various factors, such as the size of the available training data, the complexity of the language tasks to be performed, and computational resource constraints. Models may vary in size from millions to billions of parameters, with larger models generally capable of more nuanced language understanding and generation but requiring significantly more computational power to train and operate.

In accordance with one or more embodiments, training module 418, when used for LLMs, is configured to adjust the model's parameters through exposure to training data. This process utilizes optimization algorithms, such as stochastic gradient descent, to minimize the difference between the model's predictions and the actual desired outputs. The training process is computationally intensive, often requiring specialized hardware, such as Graphics Processing Units (“GPUs”) or Tensor Processing Units (“TPUs”), to manage the large volumes of data and the complexity of the model calculations. During training, techniques, such as dropout and layer normalization, are used to improve model generalization and prevent overfitting (i.e., when a model learns the detail and noise in the training data to the extent that it negatively impacts the model's performance on new data).

In accordance with one or more embodiments, evaluation and/or tuning module 422 assesses the performance of LLMs using metrics, such as perplexity, accuracy, and F1 score, depending on the specific language tasks. Evaluation may involve comparing the model's output against a set of labeled validation data, providing insight into how well the model has learned to perform tasks, such as text classification, question answering, or text generation. Tuning involves adjusting model parameters or training strategies based on evaluation outcomes to improve performance. This may include hyperparameter tuning, where parameters that govern the training process, such as learning rate or batch size, are adjusted.

In accordance with one or more embodiments, inference module 424, in the context of LLMs, is responsible for generating predictions or responses based on new, unseen data. This process involves feeding the input data through the trained model to produce an output. Inference can be used for a variety of applications, including translating text, generating human-like responses in a chatbot, or summarizing articles.

Another type of generative model is a large multimodal model (“LMM”). An LMM is an advanced ML model capable of processing and generating data across multiple modalities, such as text, images, audio, and video. These models integrate diverse datasets during training to learn the underlying distribution of different data types, enabling them to produce outputs that reflect a comprehensive understanding of the input data. These models can be used for numerous applications, such as image captioning, text-to-image generation, image-to-text generation, visual question answering, and more, where understanding the relationship between different data types is crucial. By leveraging diverse datasets during training, LMMs learn to create coherent and contextually relevant outputs across various modalities, enhancing their utility in complex, real-world scenarios.

The architecture of LLMs combines elements from different neural network designs to handle diverse data types effectively. For example, convolutional neural networks (“CNNs”) are often used for processing visual data, while transformer networks handle textual data, enabling the model to extract and synthesize features from both images and text. This integration results in outputs that accurately represent the input data, reflecting a deep understanding of both modalities. The transformer architecture, known for its ability to manage sequential data, is frequently adapted to work alongside CNNs, allowing these models to benefit from the strengths of each neural network type.

In at least some instances, the self-attention mechanism, a cornerstone of transformer networks, is integral to the functioning of LMMs. It enables the model to weigh the importance of different elements within an input sequence, regardless of their position, allowing it to capture intricate relationships between various data types. For example, in an image captioning task, the model can associate specific visual features with corresponding descriptive text, enhancing the coherence and accuracy of the generated captions. By assigning scores to relationships between elements, the self-attention mechanism highlights the most relevant connections, enabling the model to focus on the most informative parts of the input data and perform complex multimodal tasks effectively.

In LMMs, data preprocessing is a step that ensures the input data is in a suitable format for the model to process. This involves tasks, such as tokenization for text data, where the text is broken down into manageable pieces, and feature extraction for image data, where key visual elements are identified and encoded. By standardizing and normalizing different data types, preprocessing reduces the complexity of the input space, enabling the model to treat similar elements consistently. Effective preprocessing is essential for the model to integrate information from various modalities and produce accurate, meaningful outputs.

Training LMMs involves optimizing their parameters through exposure to diverse datasets that include paired data from different modalities. This computationally intensive process often requires specialized hardware, like GPUs or TPUs, to manage the large volumes of data and the complexity of the model calculations. Techniques, such as dropout and layer normalization, are employed to improve model generalization and prevent overfitting. By iteratively adjusting the model's parameters, the training process enables the model to learn underlying patterns and relationships within the data, enhancing its ability to generate coherent and contextually relevant outputs across different modalities.

Evaluation and/or tuning of LMMs are conducted using various metrics tailored to the specific tasks they are designed to perform. For example, Bilingual Evaluation Understudy scores are used for text generation tasks, while accuracy is commonly applied for visual recognition tasks to assess performance. Tuning involves adjusting hyperparameters and refining training strategies based on evaluation results to enhance the model's effectiveness. This iterative process ensures that the model can perform a wide range of multimodal tasks with high accuracy and relevance, making it a versatile tool for applications requiring the integration of different types of data.

Large multimodal models represent a significant advancement in ML by leveraging sophisticated architectures that combine different neural network types and apply self-attention mechanisms. This enables them to perform complex tasks that require understanding and synthesizing information from diverse data types. Effective preprocessing, rigorous training, and thorough evaluation are crucial to their success, allowing these models to generate coherent and contextually relevant outputs across a wide range of applications.

In accordance with one or more embodiments, other types of models besides LLMs and LMMs belong to the broad category of generative models. For example, stochastic models directly incorporate randomness into their structure, making them inherently generative, for they can produce a diverse set of outputs for a given input. Generative Adversarial Networks (“GANs”) learn to generate new data that is indistinguishable from the data they were trained on, using a dual-network architecture that involves a generative component. Variational Autoencoders (“VAEs”) are designed for generating new data points by learning a distribution of some input data, by encoding inputs into a latent space, and/or by generating outputs by sampling from a latent space. An example VAE is therefore inherently generative. Sequence-to-sequence models are generative in nature when used with sampling strategies. Although this list of generative model types is not exhaustive, it illustrates the broad use of the term generative model beyond LLMs.

Although generative models can be leveraged for classification tasks, they inherently operate on principles of randomness, leading to a spectrum of possible outcomes in response to identical inputs. Unlike deterministic models that yield a consistent result whenever the same input is given, generative models use the randomness in the data they are trained on to both mimic and diversify from the training data. This diversity makes generative models ideal for generating new and varied data points as well as for tasks that require creativity and novelty. However, a reliance on randomness creates a trade-off between predictability and flexibility for generative models, potentially making them less predictable in scenarios where uniform outcomes may be expected such as classification tasks.

### 9. Computer Networks and Cloud Networks

In one or more embodiments, a computer network provides connectivity among a set of nodes. The nodes may be local to and/or remote from each other. The nodes are connected by a set of links. Examples of links include a coaxial cable, an unshielded twisted cable, a copper cable, an optical fiber, and a virtual link.

A subset of nodes implements the computer network. Examples of such nodes include a switch, a router, a firewall, and a network address translator (“NAT”). Another subset of nodes uses the computer network. Such nodes (also referred to as “hosts”) may execute a client process and/or a server process. A client process makes a request for a computing service (such as, execution of a particular application, and/or storage of a particular amount of data). A server process responds by executing the requested service and/or returning corresponding data.

A computer network may be a physical network, including physical nodes connected by physical links. A physical node is any digital device. A physical node may be a function-specific hardware device, such as a hardware switch, a hardware router, a hardware firewall, and a hardware NAT. Additionally, or alternatively, a physical node may be a generic machine that is configured to execute various virtual machines and/or applications performing respective functions. A physical link is a physical medium connecting two or more physical nodes. Examples of links include a coaxial cable, an unshielded twisted cable, a copper cable, and an optical fiber.

A computer network may be an overlay network. An overlay network is a logical network implemented on top of another network (such as, a physical network). Each node in an overlay network corresponds to a respective node in the underlying network. Hence, each node in an overlay network is associated with both an overlay address (to address to the overlay node) and an underlay address (to address the underlay node that implements the overlay node). An overlay node may be a digital device and/or a software process (such as, a virtual machine, an application instance, or a thread) A link that connects overlay nodes is implemented as a tunnel through the underlying network. The overlay nodes at either end of the tunnel treat the underlying multi-hop path between them as a single logical link. Tunneling is performed through encapsulation and decapsulation.

In an embodiment, a client may be local to and/or remote from a computer network. The client may access the computer network over other computer networks, such as a private network or the Internet. The client may communicate requests to the computer network using a communications protocol, such as Hypertext Transfer Protocol (“HTTP”). The requests are communicated through an interface, such as a client interface (such as a web browser), a program interface, or an application programming interface (“API”).

In an embodiment, a computer network provides connectivity between clients and network resources. Network resources include hardware and/or software configured to execute server processes. Examples of network resources include a processor, a data storage, a virtual machine, a container, and/or a software application. Network resources are shared amongst multiple clients. Clients request computing services from a computer network independently of each other. Network resources are dynamically assigned to the requests and/or clients on an on-demand basis.

Network resources assigned to each request and/or client may be scaled up or down based on, for example, (a) the computing services requested by a particular client, (b) the aggregated computing services requested by a particular tenant, and/or (c) the aggregated computing services requested of the computer network. Such a computer network may be referred to as a “cloud network.”

In an embodiment, a service provider provides a system via a cloud network to one or more end users. Various service models may be implemented by the cloud network, including but not limited to Software-as-a-Service (“SaaS”), Platform-as-a-Service (“PaaS”), and Infrastructure-as-a-Service (“IaaS”). In SaaS, a service provider provides end users the capability to use the service provider's applications, which are executing on the network resources. In PaaS, the service provider provides end users the capability to deploy custom applications onto the network resources. The custom applications may be created using programming languages, libraries, services, and tools supported by the service provider. In IaaS, the service provider provides end users the capability to provision processing, storage, networks, and other fundamental computing resources provided by the network resources. Any arbitrary applications, including an operating system, may be deployed on the network resources.

In an embodiment, various deployment versions of a system may be implemented by a computer network, including but not limited to a private cloud, a public cloud, and a hybrid cloud. In a private cloud, network resources are provisioned for exclusive use by a particular group of one or more entities (the term “entity” as used herein refers to a corporation, organization, person, or other entity). The network resources may be local to and/or remote from the premises of the particular group of entities. In a public cloud, cloud resources are provisioned for multiple entities that are independent from each other (also referred to as “tenants” or “customers”). The computer network and the network resources thereof are accessed by clients corresponding to different tenants. Such a computer network may be referred to as a “multi-tenant computer network.” Several tenants may use a same particular network resource at different times and/or at the same time. The network resources may be local to and/or remote from the premises of the tenants. In a hybrid cloud, a computer network comprises a private cloud and a public cloud. An interface between the private cloud and the public cloud allows for data and application portability. Data stored at the private cloud and data stored at the public cloud may be exchanged through the interface. Applications implemented at the private cloud and applications implemented at the public cloud may have dependencies on each other. A call from an application at the private cloud to an application at the public cloud (and vice versa) may be executed through the interface.

In an embodiment, tenants of a multi-tenant computer network are independent of each other. For example, a business or operation of one tenant may be separate from a business or operation of another tenant. Different tenants may demand different network requirements for the computer network. Examples of network requirements include processing speed, amount of data storage, security requirements, performance requirements, throughput requirements, latency requirements, resiliency requirements, Quality of Service (“QoS”) requirements, tenant isolation, and/or consistency. The same computer network may need to implement different network requirements demanded by different tenants.

In one or more embodiments, in a multi-tenant computer network, tenant isolation is implemented to ensure that the applications and/or data of different tenants are not shared with each other. Various tenant isolation approaches may be used.

In an embodiment, each tenant is associated with a tenant ID. Each network resource of the multi-tenant computer network is tagged with a tenant ID. A tenant is permitted access to a particular network resource only if the tenant and the particular network resources are associated with a same tenant ID.

In an embodiment, each tenant is associated with a tenant ID. Each application, implemented by the computer network, is tagged with a tenant ID. Additionally, or alternatively, each data structure and/or dataset, stored by the computer network, is tagged with a tenant ID. A tenant is permitted access to a particular application, data structure, and/or dataset only if the tenant and the particular application, data structure, and/or dataset are associated with a same tenant ID.

As an example, each database implemented by a multi-tenant computer network may be tagged with a tenant ID. Only a tenant associated with the corresponding tenant ID may access data of a particular database. As another example, each entry in a database implemented by a multi-tenant computer network may be tagged with a tenant ID. Only a tenant associated with the corresponding tenant ID may access data of a particular entry. However, the database may be shared by multiple tenants.

In an embodiment, a subscription list indicates which tenants have authorization to access which applications. For each application, a list of tenant IDs of tenants authorized to access the application is stored. A tenant is permitted access to a particular application only if the tenant ID of the tenant is included in the subscription list corresponding to the particular application.

In an embodiment, network resources (such as digital devices, virtual machines, application instances, and threads) corresponding to different tenants are isolated to tenant-specific overlay networks maintained by the multi-tenant computer network. As an example, packets from any source device in a tenant overlay network may only be transmitted to other devices within the same tenant overlay network. Encapsulation tunnels are used to prohibit any transmissions from a source device on a tenant overlay network to devices in other tenant overlay networks. Specifically, the packets, received from the source device, are encapsulated within an outer packet. The outer packet is transmitted from a first encapsulation tunnel endpoint (in communication with the source device in the tenant overlay network) to a second encapsulation tunnel endpoint (in communication with the destination device in the tenant overlay network). The second encapsulation tunnel endpoint decapsulates the outer packet to obtain the original packet transmitted by the source device. The original packet is transmitted from the second encapsulation tunnel endpoint to the destination device in the same particular overlay network.

### 10. Microservice Applications

According to one or more embodiments, the techniques described herein are implemented in a microservice architecture. A microservice in this context refers to software logic designed to be independently deployable, having endpoints that may be logically coupled to other microservices to build a variety of applications, for example, by logically coupling a system to a software logic endpoint. Applications built using microservices are distinct from monolithic applications, which are designed as a single fixed unit and generally comprise a single logical executable. With microservice applications, different microservices are independently deployable as separate executables. Microservices may communicate using HyperText Transfer Protocol (“HTTP”) messages and/or according to other communication protocols via API endpoints. Microservices may be managed and updated separately, written in different languages, and be executed independently from other microservices.

Microservices provide flexibility in managing and building applications. Different applications may be built by connecting different sets of microservices without changing the source code of the microservices. Thus, the microservices act as logical building blocks that may be arranged in a variety of ways to build different applications. Microservices may provide monitoring services that notify a microservices manager (such as If-This-Then-That (“IFTTT”), Zapier, or Oracle Self-Service Automation (“OSSA”)) when trigger events from a set of trigger events exposed to the microservices manager occur. Microservices exposed for an application may additionally, or alternatively, provide action services that perform an action in the application (controllable and configurable via the microservices manager by passing in values, connecting the actions to other triggers and/or data passed along from other actions in the microservices manager) based on data received from the microservices manager. The microservice triggers and/or actions may be chained together to form recipes of actions that occur in optionally different applications that are otherwise unaware of or have no control or dependency on each other. These managed applications may be authenticated or plugged in to the microservices manager, for example, with user-supplied application credentials to the manager, without requiring reauthentication each time the managed application is used alone or in combination with other applications.

In one or more embodiments, microservices may be connected via a GUI. For example, microservices may be displayed as logical blocks within a window, frame, or other element of a GUI. A user may drag and drop microservices into an area of the GUI used to build an application. The user may connect the output of one microservice into the input of another microservice using directed arrows or any other GUI element. The application builder may run verification tests to confirm that the output and inputs are compatible (e.g., by checking the datatypes, size restrictions, etc.)

### Triggers

The techniques described above may be encapsulated into a microservice according to one or more embodiments. In other words, a microservice may trigger a notification (into the microservices manager for optional use by other plugged in applications, herein referred to as the “target” microservice) based on the above techniques and/or may be represented as a GUI block and connected to one or more other microservices. The trigger condition may include absolute or relative thresholds for values and/or absolute or relative thresholds for the amount or duration of data to analyze, such that the trigger to the microservices manager occurs whenever a plugged-in microservice application detects that a threshold is crossed. For example, a user may request a trigger into the microservices manager when the microservice application detects a value has crossed a triggering threshold.

In one embodiment, the trigger, when satisfied, might output data for consumption by the target microservice. In another embodiment, the trigger, when satisfied, outputs a binary value indicating the trigger has been satisfied or outputs the name of the field or other context information for which the trigger condition was satisfied. Additionally, or alternatively, the target microservice may be connected to one or more other microservices such that an alert is input to the other microservices. Other microservices may perform responsive actions based on the above techniques, including, but not limited to, deploying additional resources, adjusting system configurations, and/or generating GUIs.

### Actions

In one or more embodiments, a plugged-in microservice application may expose actions to the microservices manager. The exposed actions may receive, as input, data or an identification of a data object or location of data that causes data to be moved into a data cloud.

In one or more embodiments, the exposed actions may receive, as input, a request to increase or decrease existing alert thresholds. The input might identify existing in-application alert thresholds and whether to increase, decrease, or delete the threshold. Additionally, or alternatively, the input might request the microservice application to create new in-application alert thresholds. The in-application alerts may trigger alerts to the user while logged into the application or may trigger alerts to the user using default or user-selected alert mechanisms available within the microservice application itself rather than through other applications plugged into the microservices manager.

In one or more embodiments, the microservice application may generate and provide an output based on input that identifies, locates, or provides historical data and defines the extent or scope of the requested output. The action, when triggered, causes the microservice application to provide, store, or display the output, for example, as a data model or as aggregate data that describes a data model.

### 11. Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (“ASICs”), field programmable gate arrays (“FPGAs”), or network processing units (“NPUs”) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

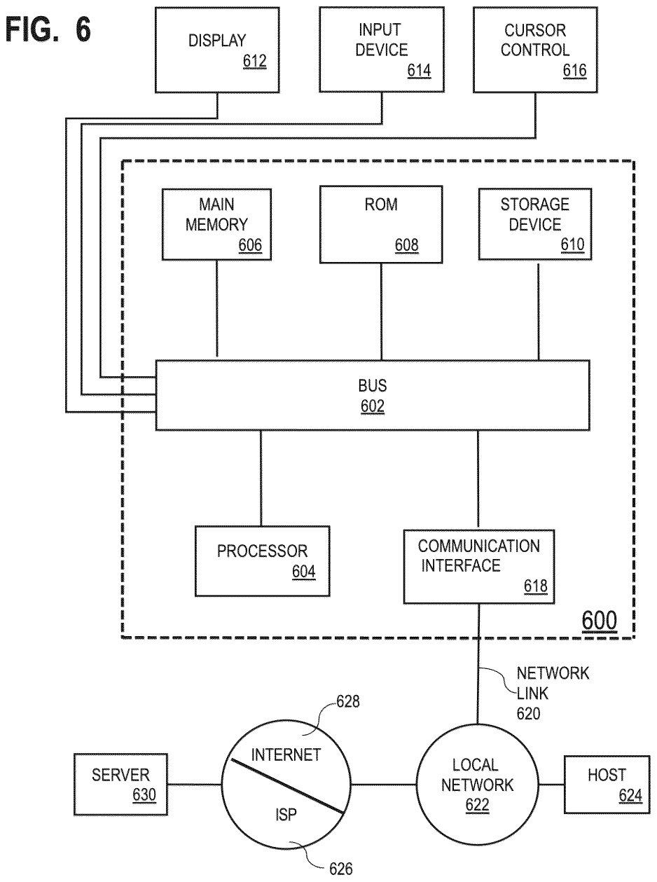

For example, FIG. 6 is a block diagram that illustrates a computer system 600 upon which an embodiment of the disclosure may be implemented. Computer system 600 includes a bus 602 or other communication mechanism for communicating information, and a hardware processor 604 coupled with bus 602 for processing information. Hardware processor 604 may be, for example, a general-purpose microprocessor.

Computer system 600 also includes a main memory 606, such as a random access memory (“RAM”) or other dynamic storage device, coupled to bus 602 for storing information and instructions to be executed by processor 604. Main memory 606 also may be used for storing temporary variables or other intermediate information during execution of instructions to be executed by processor 604. Such instructions, when stored in non-transitory storage media accessible to processor 604, render computer system 600 into a special-purpose machine that is customized to perform the operations specified in the instructions.

Computer system 600 further includes a read-only memory (“ROM”) 608 or other static storage device coupled to bus 602 for storing static information and instructions for processor 604. A storage device 610, such as a magnetic disk, optical disk, or a Solid State Drive (“SSD”) is provided and coupled to bus 602 for storing information and instructions.

Computer system 600 may be coupled via bus 602 to a display 612, such as a cathode ray tube (“CRT”), for displaying information to a computer user. An input device 614, including alphanumeric and other keys, is coupled to bus 602 for communicating information and command selections to processor 604. Another type of user input device is cursor control 616, such as a mouse, a trackball, or cursor direction keys for communicating direction information and command selections to processor 604 and for controlling cursor movement on display 612. This input device typically has two degrees of freedom in two axes, a first axis (e.g., x) and a second axis (e.g., y), that allows the device to specify positions in a plane.

Computer system 600 may implement the techniques described herein using customized hard-wired logic, one or more ASICs or FPGAs, firmware and/or program logic which in combination with the computer system causes or programs computer system 600 to be a special-purpose machine. According to one embodiment, the techniques herein are performed by computer system 600 in response to processor 604 executing one or more sequences of one or more instructions contained in main memory 606. Such instructions may be read into main memory 606 from another storage medium, such as storage device 610. Execution of the sequences of instructions contained in main memory 606 causes processor 604 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 610. Volatile media includes dynamic memory, such as main memory 606. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (“CAM”), and ternary content-addressable memory (“TCAM”).

Storage media is distinct from but may be used in conjunction with transmission media. Transmission media participates in transferring information between storage media. For example, transmission media includes coaxial cables, copper wire and fiber optics, including the wires that comprise bus 602. Transmission media can also take the form of acoustic or light waves, such as those generated during radio-wave and infra-red data communications.

Various forms of media may be involved in carrying one or more sequences of one or more instructions to processor 604 for execution. For example, the instructions may initially be carried on a magnetic disk or solid state drive of a remote computer. The remote computer can load the instructions into its dynamic memory and send the instructions over a telephone line using a modem. A modem local to computer system 600 can receive the data on the telephone line and use an infra-red transmitter to convert the data to an infra-red signal. An infra-red detector can receive the data carried in the infra-red signal and appropriate circuitry can place the data on bus 602. Bus 602 carries the data to main memory 606, from which processor 604 retrieves and executes the instructions. The instructions received by main memory 606 may optionally be stored on storage device 610 either before or after execution by processor 604.

Computer system 600 also includes a communication interface 618 coupled to bus 602. Communication interface 618 provides a two-way data communication coupling to a network link 620 that is connected to a local network 622. For example, communication interface 618 may be an integrated services digital network (“ISDN”) card, cable modem, satellite modem, or a modem to provide a data communication connection to a corresponding type of telephone line. As another example, communication interface 618 may be a local area network (“LAN”) card to provide a data communication connection to a compatible LAN. Wireless links may also be implemented. In any such implementation, communication interface 618 sends and receives electrical, electromagnetic or optical signals that carry digital data streams representing various types of information.

Network link 620 typically provides data communication through one or more networks to other data devices. For example, network link 620 may provide a connection through local network 622 to a host computer 624 or to data equipment operated by an Internet Service Provider (ISP) 626. ISP 626 in turn provides data communication services through the world wide packet data communication network now commonly referred to as the “Internet” 628. Local network 622 and Internet 628 both use electrical, electromagnetic or optical signals that carry digital data streams. The signals through the various networks and the signals on network link 620 and through communication interface 618, which carry the digital data to and from computer system 600, are example forms of transmission media.

Computer system 600 can send messages and receive data, including program code, through the network(s), network link 620 and communication interface 618. In the Internet example, a server 630 might transmit a requested code for an application program through Internet 628, ISP 626, local network 622 and communication interface 618.

The received code may be executed by processor 604 as it is received, and/or stored in storage device 610, or other non-volatile storage for later execution.

### 12. Miscellaneous; Extensions

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, one or more non-transitory computer readable storage media comprises instructions which, when executed by one or more hardware processors, cause performance of any of the operations described herein and/or recited in any of the claims.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. A method, comprising:
accessing source audio data comprising digital recordings of one or more voices;
performing digital signal processing of the source audio data, at least by enriching the source audio data with an added audio element, to obtain enriched audio data;
training a machine learning model using the enriched audio data as training data; and
applying the machine learning model to unlabeled audio data, to obtain a transcription of the unlabeled audio data;
wherein the method is performed by at least one device including a hardware processor.

2. The method of claim 1:
wherein the source audio data comprises first medical audio data obtained in a first medical setting; and
wherein the unlabeled audio data comprises second medical audio data obtained in a second medical setting.

3. The method of claim 1, wherein enriching the source audio data with the added audio element comprises adding noise to the source audio data.

4. The method of claim 1, wherein enriching the source audio data with the added audio element comprises replacing a voice, of the one or more voices, with a variation of the voice.

5. The method of claim 1, wherein enriching the source audio data with the added audio element comprises adding one or more background sound effects to the source audio data.

6. The method of claim 5:
wherein the source audio data comprises medical audio data obtained in a medical setting; and
wherein the one or more background sound effects comprises a medical background sound.

7. The method of claim 1, wherein enriching the source audio data with the added audio element comprises adjusting a speed of the source audio data.

8. The method of claim 1, further comprising:
generating a first transcription of a first digital recording in the source audio data, to obtain a first audio-transcription pair;
determining a first confidence level associated with the first audio-transcription pair;
responsive to determining that the first confidence level satisfies a first threshold confidence criterion: fine-tuning a first set of layers of the machine learning model using the first audio-transcription pair;
generating a second transcription of a second digital recording in the source audio data, to obtain a second audio-transcription pair;
determining a second confidence level associated with the second audio-transcription pair;
responsive to determining that second confidence level does not satisfy the first threshold confidence criterion: refraining from fine-tuning the machine learning model using the second audio-transcription pair.

9. The method of claim 8, further comprising:
responsive to determining that the second confidence level does not satisfy the first threshold confidence criterion: refraining from using the second audio-transcription pair to train the machine learning model.

10. The method of claim 8, further comprising:
generating a third transcription of a third digital recording in the source audio data, to obtain a third audio-transcription pair;
determining a third confidence level associated with the third audio-transcription pair;
responsive to determining that the third confidence level satisfies a second threshold confidence criterion: fine-tuning a second set of layers of the machine learning model using the third audio-transcription pair;
wherein the second threshold confidence criterion indicates higher confidence than the first threshold confidence criterion;
wherein the first confidence level associated with the first audio-transcription pair does not satisfy the second threshold confidence criterion;
wherein the second set of layers comprises more layers than the first set of layers.

11. The method of claim 1, further comprising:
generating a plurality of transcriptions of respective digital recordings in the source audio data, to obtain a corresponding plurality of audio-transcription pairs;
wherein training the machine learning model is performed using at least a subset of the plurality of audio-transcription pairs.

12. The method of claim 11, further comprising:
determining a confidence level associated with an audio-transcription pair in the plurality of audio-transcription pairs, at least by:
obtaining a first logit associated with a first token generated based on the audio-transcription pair;
computing, based on the first logit, a first confidence probability for the first token;
obtaining a second logit associated with a second token generated based on the audio-transcription pair;
computing, based on the second logit, a second confidence probability for the second token;
computing the confidence level based at least in part on the first confidence probability for the first token and the second confidence probability for the second token.

13. The method of claim 11, further comprising:
determining a plurality of confidence levels associated, respectively, with the plurality of audio-transcription pairs;
wherein enriching the source audio data with the added audio element is performed on audio-transcription pairs that satisfy a threshold confidence criterion.

14. The method of claim 1, wherein training the machine learning model uses a loss function that constrains changes made from a first iteration of the machine learning model to a second iteration of the machine learning model.

15. The method of claim 14, further comprising:
generating a plurality of transcriptions of respective digital recordings in the source audio data, to obtain a corresponding plurality of audio-transcription pairs;
determining a plurality of confidence levels associated, respectively, with the plurality of audio-transcription pairs;
wherein the loss function comprises a plurality of weights applied, respectively, to the plurality of audio-transcription pairs based on the plurality of confidence levels.

16. The method of claim 1, further comprising:
determining a change in error rate based on comparing a previous error rate to an error rate determined by comparing the transcription of the unlabeled audio data to a ground truth.

17. One or more non-transitory computer readable media comprising instructions which, when executed by one or more hardware processors, cause performance of operations comprising:
accessing source audio data comprising digital recordings of one or more voices;
generating a plurality of transcriptions of respective digital recordings in the source audio data, to obtain a corresponding plurality of audio-transcription pairs;
determining a plurality of confidence levels associated, respectively, with the plurality of audio-transcription pairs;
wherein a first confidence level in the plurality of confidence levels, associated with a first audio-transcription pair in the plurality of audio-transcription pairs, satisfies both (a) a first threshold confidence criterion and (b) a second threshold confidence criterion that indicates higher confidence than the first threshold confidence criterion;
wherein a second confidence level in the plurality of confidence levels, associated with a second audio-transcription pair in the plurality of audio-transcription pairs, satisfies the first threshold confidence criterion and does not satisfy the second threshold confidence criterion;
wherein a third confidence level in the plurality of confidence levels, associated with a third audio-transcription pair in the plurality of audio-transcription pairs, does not satisfy the first threshold confidence criterion and does not satisfy the second threshold confidence criterion;
performing digital signal processing of a subset of the source audio data associated with audio-transcription pairs that satisfy at least the first threshold confidence criterion, at least by enriching the subset of the source audio data with an added audio element, to obtain enriched audio data;
training a machine learning model using the enriched audio data as training data, wherein training the machine learning model comprises:
based on the first confidence level satisfying both the first threshold confidence criterion and the second threshold confidence criterion: fine-tuning a first set of layers of the machine learning model using the first audio-transcription pair;
based on the second confidence level satisfying the first threshold confidence criterion and not satisfying the second threshold confidence criterion: fine-tuning a second set of layers of the machine learning model using the second audio-transcription pair, wherein the second set of layers comprises fewer layers than the first set of layers; and
based on the third confidence level not satisfying the first threshold confidence criterion and not satisfying the second threshold confidence criterion: refraining from using the third audio-transcription pair to train the machine learning model; and
applying the machine learning model to unlabeled audio data, to obtain a transcription of the unlabeled audio data.

18. The one or more media of claim 17:
wherein training the machine learning model uses a loss function that constrains changes made from a first iteration of the machine learning model to a second iteration of the machine learning model.

19. The one or more media of claim 18, wherein the loss function comprises a plurality of weights applied, respectively, to the plurality of audio-transcription pairs based on the plurality of confidence levels.

20. A system comprising:
one or more hardware processors;
one or more non-transitory computer-readable media; and
program instructions stored on the one or more non-transitory computer-readable media which, when executed by one or more hardware processors, cause the system to perform operations comprising:
accessing source audio data comprising digital recordings of one or more voices;
generating transcriptions of the source data audio and determining associated confidence levels;
selecting selected source audio data of the source audio data based on the selected source audio data having an associated confidence level that satisfies a threshold criterion;
performing offline digital signal processing of the selected source audio data, at least by enriching the selected source audio data with an added audio element, to obtain enriched audio data;
training a machine learning model using the enriched audio data as training data; and
applying the machine learning model to unlabeled audio data, to obtain a transcription of the unlabeled audio data.
